(19) United States
(12) Patent Application Publication
(10) Pub. No.: US 2022/0387521 A1
(43) Pub. Date: Dec. 8, 2022

(54) METHODS FOR PREVENTING AND/OR TREATING GERD, HEARTBURN AND/OR GASTROESOPHAGEAL ACID REFLUX

(71) Applicant: LESROF LLC, ST. LOUIS PARK, MN (US)

(72) Inventors: MIRELL NAOMI LEFKOWITZ, ST. LOUIS PARK, MN (US); VICTOR A. PERONI, CHARDON, OH (US); SEAN FARMER, FT. LAUDERDALE, FL (US); ANDREW R. LEFKOWITZ, SOLON, OH (US)

(21) Appl. No.: 17/721,476

(22) Filed: Apr. 15, 2022

Related U.S. Application Data
(60) Provisional application No. 63/195,771, filed on Jun. 2, 2021.

(51) Int. Cl. A61K 35/742; A61K 38/48; A61K 38/12; C12N 1/20

(52) U.S. Cl. CPC A61K 35/742; A61K 38/482; A61K 38/12; C12Y 304/21062; C12Y 302/01004; C12Y 302/01026; C12N 1/20; A61K 2035/115

(57) ABSTRACT

The present invention relates to compositions and methods for treating or preventing gastroesophageal reflux disease (GERD) and related conditions. The subject invention utilizes a composition comprising digestive enzymes, one or more probiotic bacteria, and microbe-based products that can be administered to a subject in need thereof.

### CROSS-REFERENCE TO RELATED APPLICATION

This application claims priority to U.S. Provisional Patent Application No. 63/195,771, filed Jun. 2, 2021, which is incorporated herein by reference in its entirety.

### BACKGROUND OF THE INVENTION

Gastroesophageal reflux disease (GERD) is a common chronic digestive disease in which the liquid content of a stomach refluxes into the esophagus. Tissue damage occurs as a result of exposure of esophageal mucosa to gastric acid and bile as the gastro-duodenal content refluxes from the stomach into the esophagus. Specifically, the acid and bile damages the esophageal mucosa resulting in heartburn, ulcers, bleeding, and scarring, and long term complications such as Barrett's esophagus (pre-cancerous esophageal lining) and adeno-cancer of the esophagus. Within North America there is an estimated 18-28% of the people suffering from GERD.

Gastroesophageal reflux (GER), also known as acid indigestion and acid reflux, is a milder version of GERD. GERD is characterized as experiencing GER more than twice a week for a prolonged time.

Patients with GERD are recognized by both classic and atypical symptoms. Typical classic symptoms of GERD include heartburn, difficulty swallowing, coughing, wheezing, and chest pain. It is not uncommon for individuals with GERD to experience globus pharyngis. Alarm symptoms of GERD include, but are not limited to, dysphagia and odynophagia. These symptoms result from exposure of esophageal mucosa to gastric acid and bile as the gastro-duodenal content refluxes from the stomach into the esophagus.

In healthy individuals, acid backwash is prevented due to the lower esophageal sphincter. In individuals who experience GERD, the sphincter relaxes abnormally or becomes weak thereby allowing for acid uptake. Low esophageal sphincter relaxations are characterized as swift moments of lower esophageal sphincter tone inhibition that are independent of a swallow. Weakening of the lower esophageal sphincter can be due to impaired esophageal clearance, changes in pressure, or hiatal hernias.

The primary methods of diagnosing GERD include upper endoscopy, ambulatory acid (pH) probe test, esophageal manometry, and X-ray of upper digestive system. Upper endoscopy is a process in which an endoscope is placed down the throat of a patient to examine the esophagus and stomach. The endoscope allows for a biopsy to be collected and detection of esophagitis. The gold standard for GERD disease diagnosis is an ambulation acid (pH) probe test. The standard process for ambulation acid (pH) probe test starts with placing a monitor in the esophagus of a patient to determine the duration of acid regurgitation. The ambulation acid (pH) probe test is preferred due to its sensitivity and reproducibility. Esophageal manometry is a test that measures the rhythmic muscle contraction in the esophagus. Further, x-rays of the upper digestive system allow doctors to silhouette the esophagus region and assess damage that may have occurred due to GERD.

Risk factors for GERD include high body mass index (BMI), older age, pregnancy, and having preexisting diseases. Diet and lifestyle choices may make GERD worse. The acidity of food, as well as timing of eating with respect to sleep, can contribute to the likelihood of developing GERD. Rheumatoid arthritis, scleroderma, and lupus have been associated with higher odds of developing GERD. Babies who are in their first year of life also commonly have GERD due to a weak lower esophageal sphincter. Additionally, medications including asthma medications, antihistamines, calcium channel blockers, painkillers, and sedatives can increase chances of developing GERD. GERD can occur at any age and negatively impacts health-related quality of life (HRQL). It is also expensive to manage GERD in both primary and secondary care setting. Most expenses caused by GERD are due to complications associated with treatment.

In most cases the first line of management in treating GERD is lifestyle modification such as changing diet and exercise habits. However, the primary quantifiable lifestyle modification that helps with GERD is head of bed (HOB) elevation. Other actions that are to be avoided in treatment of GERD include smoking, intake of high dietary fat, and heavy alcohol consumption, which influence transient lower esophageal sphincter relaxations. Medication therapy for GERD is targeted to minimize the esophageal mucosal damage. Many individuals with GERD try over-the counter antacids, which typically include a H2 blocker and proton pump inhibitor.

If GERD is not controlled with medications and lifestyle changes, surgery such as laparoscopic antireflux surgery or LINX device implantation become necessary. Successful treatment of GERD has been associated with an increase in vitality, emotional well-being, and decrease in physical pain.

Thus, there is a need for compositions and methods for treating and/or preventing GERD, and associated conditions in a non-toxic, cost-effective manner.

### BRIEF SUMMARY OF INVENTION

The subject invention provides compositions and methods for preventing and/or treating gastroesophageal reflux disease (GERD), heartburn, and/or acid reflux using a combination of digestive enzymes, one or more species of probiotic bacteria, and nattokinase.

In certain embodiments, one or more of the probiotic bacteria produce nattokinase. Alternatively, nattokinase can be isolated from bacteria and then used in the subject compositions and methods.

These compositions and methods facilitate the cost-effective preparation and use of non-toxic compositions for the treatment and/or prevention of GERD, heartburn and/or acid reflux.

In certain embodiments, the composition can comprise one or more digestive enzymes selected from proteases, cellulases, lipases, hemicellulases, and/or invertases. In preferred embodiments, the compositions of the subject invention comprise at least one protease and at least one lipidase. In specific embodiments, the composition comprises the protease nattokinase, which is derived from Bacillus subtilis ssp. natto.

Advantageously, the composition of the subject invention facilitate, the disruption and breaking down of accumulations of animal fat and proteins in the digestive tract and, specifically, at the lower esophageal sphincter.

In certain embodiments, the probiotic bacterium can be from the genera Bacillus, Lactobacillus, Bifidobacterium, Lactococcus, Escherichia, Enterococcus, or Propionibacterium. In preferred embodiments the probiotic bacterium is a Bacillus spp., including Bacillus coagulans and Bacillus subtilis. In a preferred embodiment the probiotic is the Bacillus coagulans strain known as BC30 (ATCC PTA 6086).

In preferred embodiments, digestive enzymes, one or more probiotic bacteria, and/or microbe-based products (e.g., nattokinase) can be present in the subject composition in effective concentrations to treat and/or prevent GERD, heartburn, and/or acid reflux. The effective concentration of digestive enzymes can be determined based on the individual activity of the one or more digestive enzymes in the composition.

In preferred embodiments, the subject compositions are formulated and administered as orally-consumable products, such as, for example food items, capsules, pills, and drinkable liquids.

In preferred embodiments, the compositions and methods are used to treat and/or prevent GERD, heartburn, and/or acid reflux. In certain embodiments, the compositions can also enhance the digestion of food or beverages, act as a bacteriostatic or bactericidal agent, disrupt biofilm and/or neutralize acids. In other embodiments, the methods and compositions of the subject invention can be used to treat and/or prevent ailments other than GERD such as, for example, peptic ulcer disease, dyspepsia, stress ulcers, gastritis, and/or aspiration pneumonitis.

### DETAILED DISCLOSURE OF INVENTION

The subject invention provides materials and methods for treating and/or preventing GERD, heartburn and/or acid reflux. In particular, the subject invention provides compositions comprising digestive enzymes, one or more probiotic bacteria, and microbe-based products for use in methods of the subject invention. Advantageously, the compositions and methods of the invention are non-toxic and cost-effective.

Further described herein are approaches to treat and/or prevent GERD, heartburn, and/or acid reflux through various means, which include, for example, enhancing the breakdown of food or beverages, providing bacteriostatic or bactericidal activity, disrupting biofilm, and/or neutralizing acids. The subject invention also provides methods for treating and/or preventing ailments other than GERD, heartburn, and acid reflux, such as, for example, peptic ulcer disease, dyspepsia, stress ulcers, gastritis, and/or aspiration pneumonitis.

In preferred embodiments, the compositions of the subject invention work efficiently to breakdown accumulations in the digestive tract of fats and lipids that contribute to GERD, heartburn and acid reflux. The breakdown is particularly efficient due to the unique and advantageous combination of digestive enzymes, nattokinase and at least one probiotic. In preferred embodiments, the composition comprises a biosurfactant.

In certain embodiments, one or more of the probiotic bacteria produce nattokinase. Alternatively, nattokinase can be isolated from a bacterium and then used in the subject compositions and methods.

These compositions and methods facilitate the cost-effective preparation and use of non-toxic compositions for the treatment and/or prevention of GERD, heartburn and/or acid reflux.

In certain embodiments, the composition can comprise one or more digestive enzymes selected from proteases, cellulases, lipases, hemicellulases, and/or invertases. In preferred embodiments, the compositions of the subject invention comprise at least one protease and at least one lipidase. In specific embodiments, the composition comprises the protease nattokinase, which is derived from Bacillus subtilis ssp. natto.

Advantageously, the composition of the subject invention facilitate the disruption and breaking down of accumulations of animal fat and proteins in the digestive tract and, specifically, at the pyloric sphincter.

In certain embodiments, the probiotic bacterium can be from the genera Bacillus, Lactobacillus, Bifidobacterium, Lactococcus, Escherichia, Enterococcus, or Propionibacterium. In preferred embodiments the probiotic bacterium is a Bacillus spp., including Bacillus coagulans and Bacillus subtilis. In a preferred embodiment the probiotic is the Bacillus coagulans strain known as BC30 (ATCC PTA 6086).

In preferred embodiments, digestive enzymes, one or more probiotic bacteria, and/or microbe-based products (e.g., nattokinase) can be present in the subject composition in effective concentrations to treat and/or prevent GERD, heartburn, and/or acid reflux. The effective concentration of digestive enzymes can be determined based on the individual activity of the one or more digestive enzymes in the composition.

In certain embodiments, the probiotic bacteria can produce biosurfactants. In specific embodiments, the biosurfactant of the subject composition is a glycolipid biosurfactant or a lipopeptide biosurfactant. In specific preferred embodiments, the lipopeptide can be a surfactin. In certain embodiments, the lipopeptide is surfactin, with a structure comprising a peptide loop of seven amino acids and a hydrophobic fatty acid chain that is thirteen to fifteen carbons long. In specific embodiments, the amino acids comprise L-aspartic acid, L-glutamic acid, two L-leucine, two D-leucine, and L-valine. In preferred embodiments, the biosurfactant and/or other microbial metabolite disrupts harmful biofilm.

In preferred embodiments, the subject compositions are formulated and administered as orally-consumable products, such as, for example food items, capsules, pills, and drinkable liquids.

In preferred embodiments, the compositions and methods are used to treat and/or prevent GERD, heartburn, and/or acid reflux. In certain embodiments, the compositions can enhance the digestion of food or beverages, act as a bacteriostatic or bactericidal agent, disrupt biofilm and/or neutralize acids. In other embodiments, the methods and compositions of the subject invention can be used to treat and/or prevent ailments other than GERD such as, for example, peptic ulcer disease, dyspepsia, stress ulcers, gastritis, and/or aspiration pneumonitis.

### Selected Definitions

As used herein, the term “subject” refers to an animal, needing or desiring delivery of the benefits provided by a composition. The animal may be for example, humans pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. In some embodiments, a subject is suffering from a health condition, disease, or disorder, while in some embodiments, the subject is in a state of good health (e.g., free from injury or illness) but desires enhanced health and/or functioning of a particular organ, tissue, or body system. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

As used herein, reference to a “microbe-based composition” or “microbial-sourced composition” means a composition that comprises components that were produced as the result of the growth of microorganisms or other cell cultures. A microbe-based composition may comprise the microbes themselves, or the microbes may be separated from the broth or media in which they were cultivated, and the composition comprises residual cellular components and/or by-products of microbial growth. Preferably, the compositions according to the subject invention have been separated from the microbes. The by-products of microbial growth may be, for example, metabolites (e.g., biosurfactants), cell membrane components, synthesized proteins, and/or other cellular components.

The subject invention further provides “microbe-based products,” which are products that are to be applied in practice to achieve a desired result. The microbe-based product can be simply the microbe-based composition harvested from the microbe cultivation process.

Alternatively, the microbe-based product may comprise further ingredients that have been added. These additional ingredients can include, for example, stabilizers, buffers, and/or appropriate carriers (e.g., water or salt solutions). The microbe-based product may comprise mixtures of microbe-based compositions. The microbe-based product may also comprise one or more components of a microbe-based composition that have been processed in some way such as, but not limited to, filtering, centrifugation, lysing, drying, purification, and the like.

As used herein, “harvested” refers to removing some or all of the microbe-based composition from a growth vessel.

As used herein, an “isolated” or “purified” biosurfactant is substantially free of other compounds, such as cellular material, with which it is associated in nature or in the growth vessel. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (i.e., a growth by-product) or a substance necessary for taking part in a particular metabolic process. A metabolite can be an organic compound that is a starting material (e.g., glucose), an intermediate (e.g., acetyl-CoA), or an end product (e.g., n-butanol) of metabolism. Examples of metabolites include, but are not limited to, biosurfactants, enzymes, acids, solvents, gases, alcohols, proteins, vitamins, minerals, microelements, amino acids, and polymers.

The term “modulate” is meant to alter (increase or decrease). Such alterations are detected by standard methods known in the art such as those described herein.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

By “reference” is meant a standard or control condition.

The transitional term “comprising,” which is synonymous with “including,” or “containing,” is inclusive or open-ended and does not exclude additional elements or method steps not recited. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or is obvious from context, as used herein, the term “or” is understood to be inclusive. Unless specifically stated or is obvious from context, as used herein, the terms “a,” “an” and “the” are understood to be singular or plural.

Unless specifically stated or is obvious from context, as used herein, the term “about” is understood as within a range of normal tolerance in the art, for example within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value.

As used herein, “administering” a composition refers to delivering it to a subject such that it contacts a target or site in which the composition can have an effect on that target or site. Administration can be acute or chronic (e.g., hourly, daily, weekly, monthly, etc.) or in combination with other agents.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

Formulation and Delivery of Composition to Treat GERD, Heartburn and/or Acid Reflux

The subject invention provides compositions comprising digestive enzymes, one or more probiotic bacteria, and/or microbe-based products for use treating or preventing GERD, heartburn, and/or acid reflux. Advantageously, the compositions of the subject invention are non-toxic and cost-effective.

In certain embodiments, the composition can comprise a digestive enzyme selected from proteases, cellulases, lipases, hemicellulases, and invertases. The cellulase can be an endocellulase, exocellulase, cellobiase, oxidative cellulase, cellulose phosphorylases, or any combination thereof. In preferred embodiments, the protease can be a serine protease, cysteine protease, threonine protease, aspartic protease, glutamic protease, metalloprotease, asparagine peptide lyases or any combination thereof. In certain embodiments, the lipase can be a triglyceride lipase produced in a microbe, such as, a bacteria or yeast, and the invertase can be isolated from Saccharomyces cerevisiae or other microbes such as, for example, Rhizopus spp. or Aspergillus casiellus.

In certain embodiments the digestive enzymes can be isolated from the production source (e.g., microorganism) and individually added. Alternatively, the digestive enzymes can be synthesized by microbes, including bacteria and/or yeast, and the microorganisms can be used in the composition. In preferred embodiments, the probiotic bacteria of the subject composition can produce digestive enzymes, which can enhance the digestion of food or beverages in a subject.

In certain embodiments, the amount of digestive enzymes in the composition is 0.001 to 99% to weight (wt %), preferably 10% or more, more preferably 25 wt % or more, even more preferably 50 wt % or more. In certain embodiments, the digestive enzymes are present at more than 0.01, 0.02, 0.03, 0.05, 0.08, 0.1, 0.2, or 0.5%.

In certain embodiments, probiotic bacteria can be added to the composition, including Lactobacillus spp. (L. plantarum, L. brevis, L. paraplantarum, L. coryniformis, L. pentosus, L. lactis, L. fermentum, L. acidophilus, L. delbrueckii ssp. bulgaricus, L. helveticus, and L. kefiranofaciens), Bacillus spp. (B. coagulans and B. subtilis (including B. subtilis spp. natto)), Leuconostoc spp. (L. mesenteroides, L. citreum, and L. argentinum), Pediococcus pentosaceus, Weissella spp., Bifidobacterium spp. (B. bifidum), Streptococcus thermophiles, Lactococcus spp. (L. lactis and L. bulgaricus), Komagataeibacter xylinus, Acetobacter spp. (A. pasteurianus and A. aceti), Escherichia spp., Enterococcus spp., Propionibacterium spp., and Gluconobacter oxydans. In preferred embodiments, the probiotic bacterium is Bacillus subtilis ssp. natto and/or Bacillus coagulans. In a specific embodiment, the probiotic is BC-30.

The probiotic bacteria can be preferably present in the subject composition in therapeutically-effective amounts. In one embodiment, the probiotic bacteria can be present in a concentration sufficient to colonize a subject, preferably the gastrointestinal tract of the subject; synthesize an effective concentration of bioactive metabolites (e.g., biosurfactants, digestive enzymes, short-chain fatty acids); reduce the pH in the subject, preferably the liminal pH in the colon; and/or inhibit the growth of pathogenic microorganisms in the gastrointestinal tract, such as, for example Helicobacter pylori, and bacteria that form biofilm.

In certain embodiments, the amount of probiotic bacteria in the composition is 0.001 to 90% to weight (wt %), preferably 50% or less, more preferably 25 wt % or less, even more preferably 10 wt % or less. In certain embodiments, the probiotic bacteria are present at more than 0.01, 0.02, 0.03, 0.05, 0.08, 0.1, 0.2, or 0.5%.

In certain embodiments, the composition can comprise microbe-based products, including proteins, lipids, biosurfactants, and carbohydrates. The proteins can be enzymes, including the aforementioned digestive enzymes. In preferred embodiments, the microbe-based product can be a serine protease, specifically nattokinase. In certain embodiments, the composition comprises acid-neutralizing compounds, which can be produced by the probiotic bacteria. The acid-neutralizing compounds can be proteins, such as, for example urease and arginine deaminase, or alkaline compounds, such as, for example, ammonia.

In certain embodiments, the amount of microbe-based product in the composition is 0.001 to 90% to weight (wt %), preferably 50% or less, more preferably 25 wt % or less, even more preferably 10 wt % or less. In certain embodiments, the microbe-based product is present at more than 0.01, 0.02, 0.03, 0.05, 0.08, 0.1, 0.2, or 0.5%.

In certain embodiments, the composition can comprise biosurfactants, which are a structurally diverse group of surface-active substances produced by microorganisms, including probiotic bacteria. Biosurfactants are safe, biodegradable and can be produced with ease at low cost using selected organisms in or on renewable substrates. The biosurfactant-synthesizing microorganisms can be used directly in the compositions of the subjection invention, or biosurfactants synthesized by microorganisms can be isolated and then added to the compositions. In preferred embodiments, the biosurfactant is produced by Bacillus subtilis ssp. natto and/or Bacillus coagulans.

All biosurfactants are amphiphilic. They consist of two parts: a polar (hydrophilic) moiety and a non-polar (hydrophobic) group. Due to the amphiphilic structure, biosurfactants increase the surface area of hydrophobic, water-insoluble substances, increase the water solubility of such substances, and change the properties of bacterial cell membranes. Biosurfactants accumulate at interfaces and reduce the surface and interfacial tension between molecules of liquids, solids, and gases, leading to the formation of aggregated micellular structures in solution once the concentration reaches a critical micelle concentration (CMC).

Biosurfactants include low molecular weight glycolipids (e.g. rhamnolipids, mannosylerythritol lipids (MELs), sophorolipids, and trehalose lipids), lipopeptides (e.g. surfactin, iturin, fengycin, and lichenysin), flavolipids (FLs), fatty acids, phospholipids, and high molecular weight polymers such as lipoproteins, lipopolysaccharide-protein complexes, and polysaccharide-protein-fatty acid complexes.

Most biosurfactant-producing organisms produce biosurfactants in response to the presence of a hydrocarbon source (e.g., oils, sugar, glycerol, etc.) to facilitate uptake. Other media components, such as the concentration of iron, can affect biosurfactant production significantly. Microbial biosurfactants are produced by a variety of microorganisms such as bacteria, fungi, and yeasts. Non-limiting examples include Pseudomonas spp. (P. aeruginosa, P. putida, P. fluorescens, P. fragi, and P. syringae), Flavobacterium spp., Bacillus spp. (B. subtilis, B. pumilus, B. cereus, B. coagulans, and B. licheniformis), Campylobacter spp., Rhodococcus spp., Arthrobacter spp., Corynebacterium spp., Starmerella spp. (S. bombicola), Wickerhamomyces spp. (W. anomalus), Candida spp. (C. albicans, C. rugosa, C. tropicalis, C. lipolytica, C. glabrata, and C. torulopsis), Pichia spp. (P. anomala and P. occidentalis). The biosurfactant may be obtained by a fermentation process known in the art such as solid-state fermentation, submerged fermentation, or a combination thereof.

In specific embodiments, the subject composition comprises one or more lipopeptide biosurfactants. The one or more biosurfactants can further be chosen from: a modified form, derivative, fraction, isoform, or subtype of a biosurfactant, including forms that are naturally or artificially modified. In certain preferred embodiments, the lipopeptide is a surfactin, with a structure comprising a peptide loop of seven amino acids and a hydrophobic fatty acid chain that is thirteen to fifteen carbons long. Advantageously, the fatty acid chain allows for penetration of a cellular membrane. In specific embodiments, the amino acids comprise L-aspartic acid, L-glutamic acid, two L-leucines, two D-leucines, and L-valine.

The surfactin is preferably present in the subject composition in a therapeutically-effective amount. In one embodiment, this means the biosurfactant can be present in a critical micelle concentration (CMC). The CMC is the concentration of surfactants above which micelles will form and any additional surfactants that are added to the composition create additional micelles or are incorporated into existing micelles.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The subject compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants (e.g., magnesium stearate), wetting agents, emulsifiers, sweeteners, colorants, flavorings, aromatizers, thickeners (e.g. carbomer, gelatin, hypromellose, or sodium alginate), coatings, preservatives (e.g., Thimerosal, benzyl alcohol, polyquaternium), antioxidants (e.g., ascorbic acid, sodium metabisulfite), anti-caking agent (e.g., silicon dioxide and microcrystalline cellulose), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance, carrier or excipient use in the subject compositions may be contemplated.

In one embodiment, the composition can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

Further components can be added to the compositions as are determined by the skilled artisan such as, for example, buffers, carriers, viscosity modifiers, preservatives, flavorings, dyes and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

Methods of Treating and/or Preventing GERD and/or Other Related Ailments

The subject invention further provides a method of treating or preventing GERD, heartburn, and or acid reflux in a subject in need thereof. In certain embodiments, the subject method can also be used to treat or prevent, either directly or indirectly, peptic ulcer disease, dyspepsia, stress ulcers, gastritis, and/or aspiration pneumonitis.

The subject composition can be administered orally in order to achieve the therapeutic effects. The therapeutic effects can be, for example, systemic, local, tissue-specific, etc. depending on the specific needs of a given application of the invention.

The method can be used to enhance digestion of food or beverages in the subject. In certain embodiments, the one or more digestive enzymes can enhance digestion of food or beverages. The digestive enzymes can be a protease, cellulase, lipase, hemicellulase, and/or an invertase. The protease can enhance the digestion of proteins, specifically catalyzing proteolysis in which proteins are broken down into polypeptides or even single amino acids by cleaving peptide ponds. The cellulase can enhance digestion of cellulose and other polysaccharides, specifically catalyzing the breakdown of cellulose into monosaccharides, such as, for example, glucose. Similarly, the hemicellulase can break down hemicellulose, a type of cellulose, into monosaccharides. The cellulase can be an endocellulase, exocellulase, cellobiase, oxidative cellulase, cellulose phosphorylase, or any combination thereof. The lipase can enhance the digestion of lipids by catalyzing the hydrolysis of lipids by, for example, breaking down dietary fats into monoglycerides and fatty acids. The invertase can enhance digestion of sucrose by catalyzing the hydrolysis of sucrose into fructose and glucose. In certain embodiments, the enhanced digestion of food or beverages, specifically of food containing cellulose or hemicellulose, can prevent obstruction of the pyloric sphincter by fibrous clots.

In certain embodiments, the method can be used to neutralize gastric acids in the subject. In certain embodiments, the composition comprises acid-neutralizing compounds, which can be produced by the probiotic bacteria. In other embodiments, chemical components of the composition can comprise salts of aluminum, calcium, magnesium, or sodium. In certain embodiments, saliva production can be increased; saliva can neutralize stomach acid.

Methods of the subject invention can inhibit microbial growth, including bactericidal and bacteriostatic effects, in the subject. In certain embodiments, the compositions of the subject invention disrupt biofilm. In certain embodiments, the administration of probiotic bacteria to a subject increases competition for resources, including carbon and oxygen. The probiotic bacteria can displace other bacteria in the gastrointestinal system, including pathogenic bacteria, such as Helicobacter pylori. Further, the probiotic bacteria can produce compounds, such as, for example, butyric acid, which inhibit the growth of other microorganisms. In certain embodiments, the probiotic bacteria attach to enterocytes, inhibiting the binding of other bacteria by a process of competitive exclusion. In certain embodiments, probiotic bacteria can induce commensal microorganism in a subject to produce lactic acid or bacteriocins.

Additionally, the attachment of probiotic bacteria to enterocytes or other cells in the gastrointestinal system can initiate immune signaling events. In certain embodiments, the immune signaling can result in the synthesis of cytokines, leading to immune-induced antimicrobial activity in a subject. In certain embodiments, toll-like receptor (TLR) signaling pathways can modulate the immune activity in a subject, including TLR 2, TLR 4, TLR 5, and TLR 9 signaling. The TLR signaling can affect inflammation, such as, for example, reducing colitis and gastritis. TLR can also affect functioning of dendritic cells, B-cells, natural killer cells, and macrophages.

### Example 1: Formulation

In a specific example, the subject invention provides a composition, in capsule form, comprising a digestive enzyme complex, nattokinase, and a Bacillus coagulans probiotic.

The protease complex can comprise, or consist of, or consist essentially of, Protease (15000 HUT), Acid Stable Protease (50 SAP), Lipase (375 FIP), Cellulase (100 CU), Hemicellulase (325 HCU), and Invertase (300 SU)

In preferred embodiments, the capsule is gluten free, and contains no artificial flavors, preservatives or colorings.

We claim:

1. A composition for treating gastroesophageal reflux disease wherein said composition comprises at least one digestive enzyme, Bacillus coagulans, and nattokinase.

2. The composition of claim 1, further comprising Bacillus subtilis var. natto.

3. The composition of claim 1, further comprising one or more lipopeptides.

4. The composition of claim 3, wherein the lipopeptides are produced by Bacillus subtilis var. natto.

5. The composition of claim 3, wherein the lipopeptides are selected from surfactins, iturins, lichenysins, and fengycins.

6. The composition of claim 3, wherein the lipopeptide is a surfactin.

7. The composition of claim 3, wherein the lipopeptide is present in critical micelle concentration (CMC).

8. The composition of claim 1, wherein the digestive enzyme is a protease, a cellulase, a lipase, a hemicellulase, an invertase, or any combination thereof.

9. The composition of claim 8, wherein the protease is an acid stable protease.

10. The composition of claim 1, formulated for oral administration to a subject.

11. The composition of claim 1, wherein the probiotic is a Bacillus subtilis or Bacillus coagulans.

12. The composition of claim 11, wherein the probiotic is BC30.

13. A method for treating or preventing gastroesophageal reflux disease in a subject, the method comprising administering to the subject a therapeutically-effective amount of a composition of claim 1.

14. The method of claim 13, used to enhance digestion of food or beverages in the subject.

15. The method of claim 14, wherein the food comprises cellulose and/or hemicellulose.

16. The method of claim 13, used to neutralize gastric acids in the subject.

17. The method of claim 13, used to inhibit microbial growth in the subject.

18. The method of claim 17, wherein the pathogenic bacteria are Helicobacter pylori.

19. The method of claim 13, used to induce immune signaling events in the subject.

20. The method of claim 19, wherein the initiation of immune signaling events reduce inflammation.

21. The method of claim 20, wherein inflammation is gastritis.
